(19) United States
(12) Patent Application Publication
(10) Pub. No.: US 2016/0174908 A1
(43) Pub. Date: Jun. 23, 2016

(54) DEVICE AND METHOD FOR HIGHLIGHTING PATIENT DATA AND TRENDS

(71) Applicant: GENERAL ELECTRIC COMPANY, Schenectady, NY (US)

(72) Inventors: Renjith S. Nair, Bangalore (IN); Sahika Genc, Niskayuna, NY (US); Stephen Thomas Treacy, Menomonee Falls, WI (US); Bruce Arnold Friedman, Jasper, GA (US); David Alan Sitzman, Hubertus, WI (US); William James Balloni, Menomonee Falls, WI (US)

(21) Appl. No.: 14/735,887

(22) Filed: Jun. 10, 2015

(30) Foreign Application Priority Data
Dec. 23, 2014 (IN) 6498/CHE/2014

(51) Int. Cl. A61B 5/00; A61B 5/04; A61B 5/026; A61B 5/1455; A61B 5/0452; A61B 5/0215

(52) U.S. Cl. CPC A61B 5/742; A61B 5/0452; A61B 5/0215; A61B 5/026; A61B 5/14551; A61B 5/746; A61B 5/04012

(57) ABSTRACT

A monitoring or diagnostic device including a display having a display screen on which incoming data from sensors attached to the item or object being monitored by the device is illustrated. The incoming data is represented on the display screen to enable an individual viewing the display screen to determine the current operating condition or parameters of the item or object, such as a patient. The device and display screen can also illustrate various alarm conditions or events, as determined by the device to draw the attention of the individual to those alarm conditions. To assist in this function, the device represents the incoming data from a sensor giving rise to the alarm condition or event, as well as additional incoming data from other sensors corresponding in some manner to the alarm condition, on the display screen in a visually different or distinct manner from the rest of the incoming data not relating to the alarm condition or event.

### BACKGROUND OF THE INVENTION

The invention relates generally to monitors or displays connected to monitoring or diagnostic equipment for illustrating data about an object to which the equipment is connected, and more particularly to devices and methods for graphically displaying the data.

In monitoring or diagnostic devices that are currently utilized, the data obtained by the devices is often shown on a display connected to the device that provides a visual representation of the data in manner that can be readily assimilated by an individual viewing the data.

These types of devices are utilized in many different environments, such as in hospitals and other medical environments where patients are continuously monitored by these devices, as well as in various other manufacturing and industrial environments where the operation of various types of equipment is continuously monitored. In the example of those devices utilized in medical environments, the data obtained and displayed on the monitoring or diagnostic devices is often shown as a waveform or trends displayed on a screen of the device. For example, on a screen 102 associated with a bedside cardiac monitoring device 100 (FIG. 1), remote cardiac monitoring device or other cardiac monitoring or diagnostic equipment the waveform, such as a monochromatic electrocardiogram (ECG) waveform 18 (FIG. 2) or an arterial blood pressure (ABP) waveform 20 (FIG. 2), is displayed as a single color line graph on a black background, or other similar type of graph, plotted over time. The clinicians observing the screen 102 and the waveforms 18,20 represented thereon derive the necessary information by viewing and analyzing the waveforms 18,20.

In collecting and formatting the data from the patient for display in the waveforms 18,20 on the associated screen 102, the monitoring device 100 also generate alarms based on sensed cardiac events based on the inbuilt algorithms and set limits regarding the data represented in the waveforms 18,20. In many situations, the algorithm utilized within the monitoring device 100 is designed to graphically reflect the cardiac event 22, e.g., arrhythmia, in the ECG waveform 18, as well as in any other relevant waveform, such as the ABP waveform 20, and generate a corresponding alarm. In most situations, the alarms corresponding to the arrhythmia events 22 represented in the data in the displayed waveforms 18,20 are various types of audio and/or visual indicators generated from the monitoring device 100.

The visual alarm indicators normally include a flashing alarm icon on the screen 102 of the device 100 in or associated with bright, easily noticeable colors (usually red) adjacent to the waveform(s) 18,20 to indicate which waveform 18,20 or associated parameter has to be reviewed by the individual on an urgent basis. Most often these visual indicators of alarms are limited to highlighting numerical values which are abnormal or the name of any critical cardiac event, e.g. ASYSTOLE.

However, other diagnostic and monitoring devices have been developed that provide information in addition to the alarm condition and the numerical values and/or name of the critical cardiac event that created the alarm. Examples of these types of devices and methods are shown in US Patent Application Publication No. US2011/0138323, and U.S. Pat. Nos. 8,269,620 and 8,396,544.

However, while these devices provide displays and methods of operating the displays that are capable of organizing information relating to various cardiac alarm events or conditions for review by an individual, due to the large number of the alarm events which may be occurring at a given time, certain highly important clinical cardiac events could inadvertently be overlooked or missed. This is often referred to as alarm fatigue and results from the constant representation of the alarm events in a similar manner that can cause certain events to become “lost” in the flood of alarms and associated information represented on the display screen of the particular device. Thus, when studying the waveforms 18,20, the individuals are using their past experience to detect any anomaly in the waveforms 18,20 in a manual fashion by directly observing the waveforms 18,20. As such, when the waveforms 18,20 are viewed along with other alarms and information provided on the display, and in conjunction with any other potential distraction(s) in the immediate vicinity, there is the potential that significant clinical data present in one or more of the waveforms 18,20 can be missed.

Therefore, it is desirable to develop a monitoring or diagnostic device and display, as well as an associated method for displaying information relating to a particular alarm condition that provides the information about the alarm event or condition in a form that can be readily and easily assimilated by an observing individual and that reduces the potential for the alarm event to become lost in other alarm event data.

### BRIEF DESCRIPTION OF THE INVENTION

In the present invention, a monitoring or diagnostic device includes a display screen on which data concerning item or object being monitored by the device is illustrated. The incoming data sensed by the device is represented on the display screen to enable an individual viewing the display screen to determine the current operating condition or parameters of the object, such as a patient. The device and display screen can also illustrate various alarm conditions or events, as determined by the device from the incoming data signals received by the device from sensors attached to the item being monitored. The alarm conditions or events are triggered by the comparison or application of preset value or parameter ranges for the incoming data signals and/or inbuilt algorithms stored within the device to the incoming data signals from the sensors. The stored ranges and/or algorithm can relate to various aspects or attributes of the incoming data signals from the sensors, including but not limited to predetermined maximum and minimum values for the data signals, frequency ranges for the incoming data, as well as correlations between one set of incoming data from one sensor and another set of incoming data from another sensor, such a temporal correspondence, among others. When an alarm event or condition is determined in this manner by the device, the device modifies the operation of the display concerning the sensed data that created the alarm event or condition to draw the attention of the monitoring individual to those particular data signals. To assist in this function, the device represents the data giving rise to the alarm condition or event on the display in a visually different or distinct manner from the rest of the data outside of the alarm condition or event, such as in a different color, to visually indicate or identify to the observing individual the data displayed on the screen that directly relates to the determined alarm condition or event. In this manner, the device and display can quickly provide the individual with the required information concerning the alarm event in order for the individual to determine and address the condition which caused the alarm event to be triggered.

The device also alters the representations of other sensed parameters on the display that correspond, e.g., in time and duration, to the sensed alarm condition in a visually distinct manner as well, regardless of whether those other sensed parameters also give rise to an alarm event or condition. In this manner, the device can visually distinguish all incoming data signals relating to the sensed alarm condition or event from the remainder of the incoming data signals being displayed to enable the observing individual to quickly locate and view all data that may be relevant to the identified alarm condition or event.

It is to be understood that both the foregoing general description and the following detailed description are exemplary and explanatory and are intended to provide further explanation of the invention as claimed.

### BRIEF DESCRIPTION OF THE DRAWINGS

FIG. 1 is a schematic view of a monitoring device in accordance with an exemplary embodiment of the invention.

FIG. 2 graphically illustrates ECG and ABP waveforms as represented on a display of a prior art monitoring device.

FIG. 3 graphically illustrates ECG and ABP waveforms as represented on a display of the monitoring device in accordance with an exemplary embodiment of the invention.

FIG. 4 graphically illustrates ECG and Pulse Oximieter waveforms as represented on a display of the monitoring device in accordance with an exemplary embodiment of the invention.

FIG. 5 graphically illustrates ECG and ABP waveforms as represented on a display of the monitoring device in accordance with an exemplary embodiment of the invention.

### DETAILED DESCRIPTION OF THE INVENTION

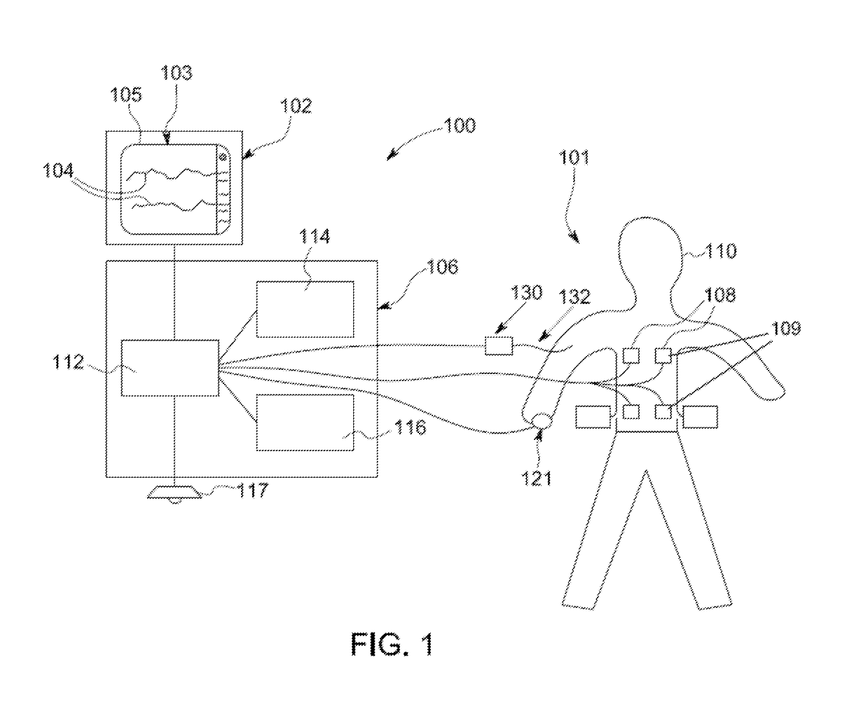

FIG. 1 illustrates an exemplary embodiment of the invention which includes a monitoring or diagnostic device 100, which can be any suitable type of monitoring device for monitoring various operating parameters of an object, machine, individual or other item 101 operably connected to the device 100. The device 100 includes a display 102 of any suitable type, such as a touch screen display, having a screen 103 thereon on which the monitoring data signals 104 regarding the object 101 connected to the device 100 can be displayed. When formed as a touch screen, the display 102 can additionally function as a user interface 105 for use in controlling the operation of the device 100, though the interface 105 can be formed as a separate component connected to the device 100, if desired.

In the exemplary embodiment of FIG. 1, the device 100 takes the form of a medical monitoring device 106 that has one or more leads or sensors 108, such as ECG electrodes 109, operably connected in any suitable manner between the medical monitoring device 106 and a patient 110 in order to monitor various vital statistics of the patient 110. In addition to the electrodes 109, the device 100 can employ additional sensors 108 used to monitor other parameters or statistics of the patient 110, such as a pulse oximeter sensor 121 and/or an invasive pressure catheter 130 and invasive pressure transducer 132 to measure the blood flow and pressure of the patient 110 for comparison with the data obtained from the ECG electrodes 109.

“Definitions” Means

The medical monitoring device 106 includes a central processing unit (CPU) 112 operably connected to the sensors 108 in order to receive and process data from the sensors 108 on the various vital statistics or parameters of specified bodily functions of the patient 110, which in the exemplary embodiment of FIGS. 1-5 relates to cardiac functions, though other bodily functions or systems are also contemplated as being within the scope of the present invention. These parameters can then be transmitted from the CPU 112 to the display 102 for presentation in a specified manner on the screen 103 of the display 102 for review by an individual monitoring the patient 110 via the display 102.

The device 100 also includes memory module 114, which can take the form of any suitable computer-readable storage media, for example a RAM module, and an electronic storage medium, component or database 116, each of which are operably connected to the CPU 112 in order to assist in the monitoring function of the device 100 using the data signals 104 supplied to the CPU 112 via the sensors 108. The device 100 also includes an audio speaker 117 for enabling the device 100 to provide audible indications of various operating characteristics of the device 100.

In addition, storage component 116 can include certain information regarding the predetermined normal or acceptable ranges for the operating parameters, vital statistics or physiological parameters for the patient 110 to which the device 100 is connected. These stored ranges can be utilized by the CPU 112 in conjunction with the incoming data signals 104 from the sensors 108 and the personal statistics of the patient 110 to determine the current vital statistics or physiological parameters of the patient 110 and whether those vital statistics or physiological parameters are outside of the predetermined normal ranges for those particular vital statistics or physiological parameters. The stored ranges for the incoming data signals 104 on the different physiological parameters and vital statistics received by the CPU 112 from the various sensors 108 can include ranges for minimum and maximum absolute values of the sensed parameters, minimum and maximum frequency ranges for the sensed physiological parameters, or any other suitable aspect of the incoming data signals 104 to be used in the determination of an alarm event or condition, such as an arrhythmia. If any sensed vital statistic or physiological parameter is outside of the stored predetermined normal ranges associated with that vital statistic or parameter, the CPU 112 determines that an alarm condition or event, such as a cardiac alarm condition or event, is occurring/has occurred, and can operate the display 100 in manner to be described to alert the individual monitoring the display 102 of the alarm condition or event, and to provide the individual with an identification of the incoming data signals 104 particularly relating to the cardiac alarm event. In addition, in association with a suitable inbuilt algorithm or other operating characteristic present within the device 100, the CPU 112 can determine the relationship between any cardiac alarm condition or event as determined by abnormal signals received from one set of incoming data signals 104, e.g., the data signals from the ECG sensors 109, with the corresponding data signals 104 received over the same time frame from one or more additional sensors 108, such as the invasive pressure catheter 130 or pulse oximeter sensor 121, to identify the sections of each of the respective data signals 104 corresponding to the cardiac alarm condition/event sensed from one or more of the incoming data signals 104 from the ECG sensors 109, catheter 130 and/or pulse oximieter sensor 121.

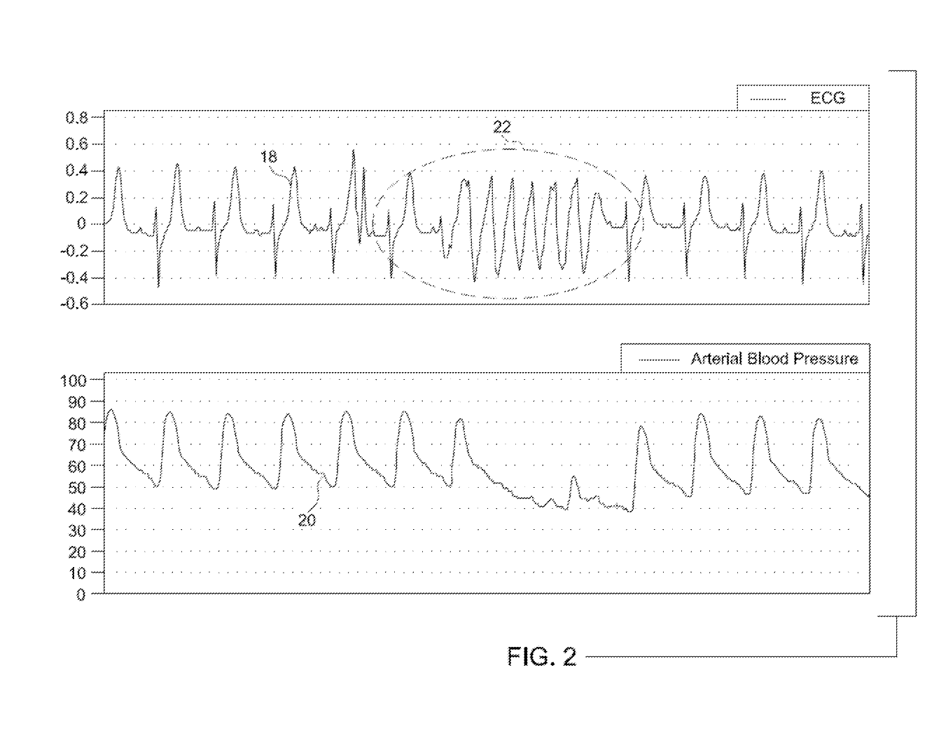

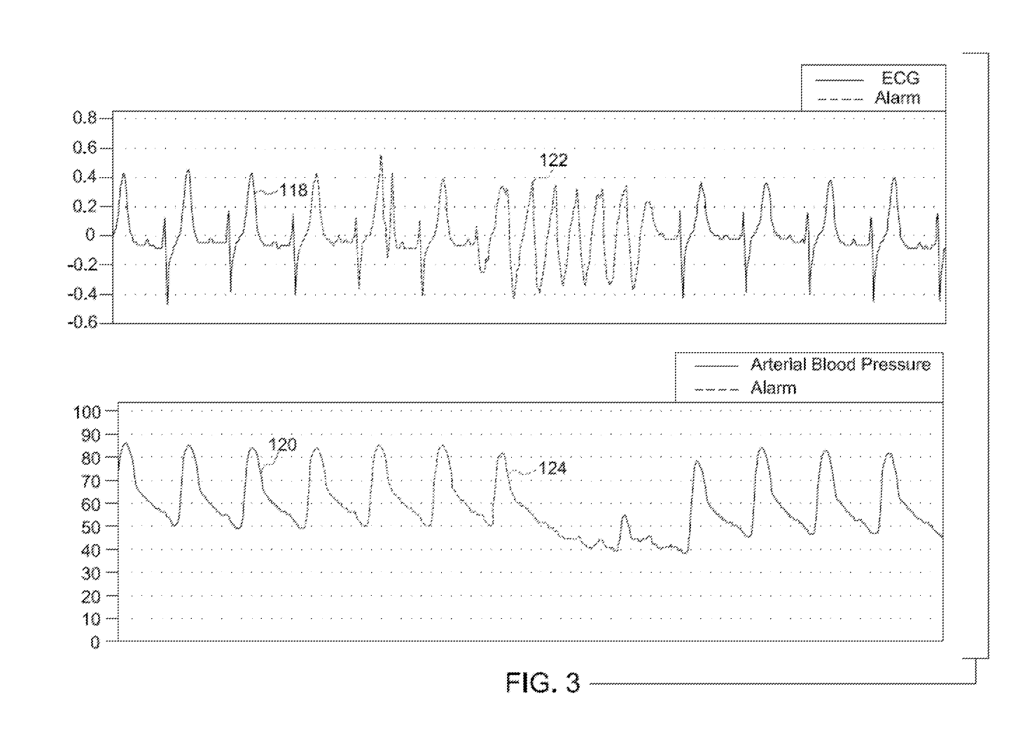

In the exemplary embodiment of FIG. 3, a waveform 118 for an ECG measurement and a waveform 120 for an ABP measurement are shown as illustrated together on the screen 103 of the display 102 as representing the monitoring data signals 104 received from the sensors 108, i.e., from the ECG electrodes 109 and invasive pressure catheter 130, respectively. The waveforms 118,120 are similar to the waveforms 18,20 of FIG. 2, with the significant difference being that the data representing the corresponding sections 122,124 of the waveforms 118,120 that are outside of the normal ranges and causing an associated alarm condition as determined by CPU 112 and/or inbuilt algorithm of the device 100 are represented differently, e.g., in a visually distinct manner, than the remainder of the waveforms 118,120 showing data within or inside the predetermined ranges. This visually distinct manner for illustrating the sections 122,124 assists in marking the important sections 122,124 of the waveform/trend 118, 120, such as the portion of the data signal 104 from the particular sensor 108 indicating an alarm condition or event represented by the sections 122,124 and its duration. In the exemplary embodiment, the sections 122,124 are identified by a visually distinct manner of indication, such as overlaying or illustrating the sections 122,124 of the waveforms 118,120 with a different color than the remainder of the waveforms 118,120, such as by providing the sections 122,124 on the display 102 in red in contrast to the normal white, yellow, blue, green or other color of the remainder of the waveforms 118, 120. However, any other suitable visually distinct manners of indication to visually separate the sections 122,124 from the remainder of the waveforms 118,120 are also contemplated, such as other color combination of the waveforms 118,120 and sections 122,124, highlighting the background of the display 102 containing the sections 122,124, bolding the waveforms 118,120 within the sections 122,124, and/or animating the sections 122,124, such as by intermittently flashing the sections 122,124, relative to the remainder of the waveforms 118,120, among others. The visually distinct highlight of the sections 122,124 on the waveforms 118,120 itself marks out the significant sections 122,124 of the waveform/trend data 104, thus aiding the individual in the detection and further investigation of alarm events represented in the data 104. This visual distinction for the waveform sections 122,124 as represented on the display 102 for the device 100 can additionally be extended to the illustration of the waveforms 118,120 and relevant sections 122,124 produced by any print devices (not shown) that are connected to the diagnostic device 100, where the sections 122,124 are represented in the printed waveform data in the same or a different visually distinct manner from the data signals 104 shown on the display 102, thereby achieving a similar function of easy detection and investigation of those sections 122,124 and their causes.

Further, in a device 100 that provides data on multiple aspects or types of vital functions of the patient 110 in a side-by-side manner on the display 102, the visually distinct manner of indications made in each corresponding section 122,124 of the waveforms 118,120 can also help in studying and comparing related multiple waveform/trend data. For example, in the ECG and ABP data 104 illustrated in waveforms 118,120 of FIG. 3 with the clinically significant sections 122,124 clearly identified, the identification of sections 122,124 in the adjacent waveforms 118,120 assists the observing individual in determining any correlation between the data signals 104 in the sections 122,124 of each waveform 118,120.

In addition, by being able to identify the clinically significant data signals 104 in the highlighted visually distinct sections 122,124 of the waveforms 118,120, among other benefits the individual viewing the display 102 is able to see a clear demarcation of the alarm event, e.g., arrhythmia, across the various comparative data signals 104 in each of the waveforms 118,120 from the start to finish of the event, and is able to the study of the variations in the each of the waveforms/trends 118,120 during, before and after the alarm event. As a result, the highlighted sections 122,124 of each of the waveform/trend 118,120 can be used for further investigations and drill-downs into the causes of the event, as well as for inclusion in relevant electronic or printed documentation on the patient 110 and for further communications such as annotations, specialist referrals etc., regarding the further treatment of the patient 110.

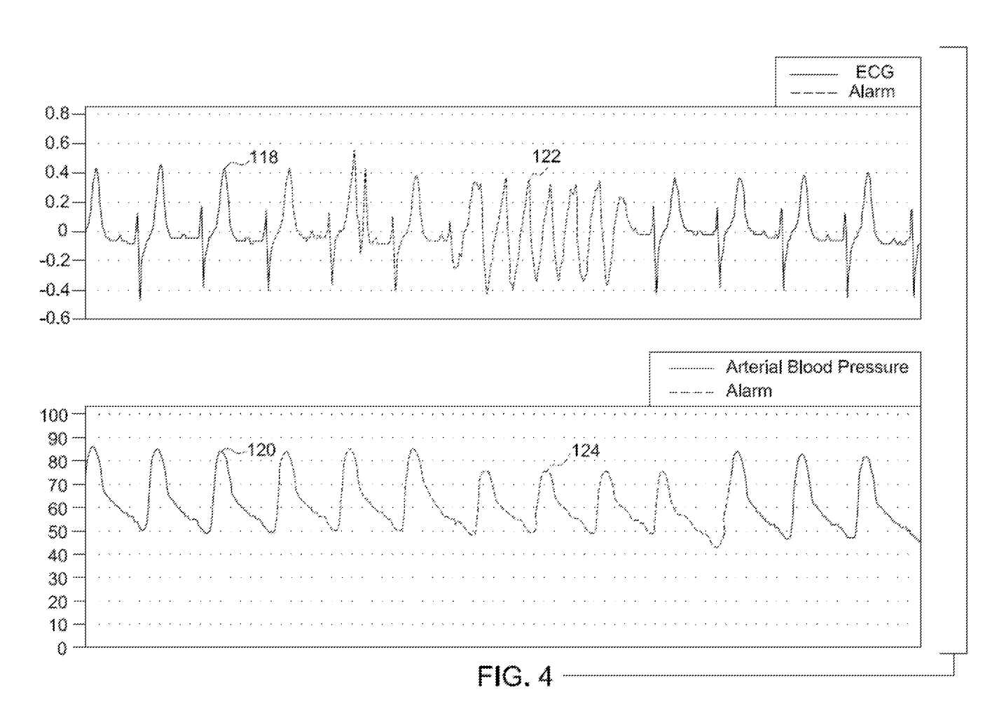

Referring now to FIG. 4, in another exemplary embodiment, as previously disclosed the device 100 also provides a visually distinct manner of displaying the various sections 122,124 of the waveforms 118,120, respectively, that correspond to an alarm, e.g., cardiac event as determined by data signals from one sensor(s) 108, i.e., the ECG sensors 109, but that do not also define a cardiac event as determined by data signals 104 from a separate sensor(s) 108, i.e., the invasive pressure catheter 130. For example, when the data signals from ECG sensors 109 are determined to represent a cardiac event in section 122 in waveform 118, CPU 112 and/or algorithm will determine and display the section 124 of waveform 120 corresponding, e.g., having a temporal correlation, to the event/section 122 in a visually distinct manner as well, which may be the same or different than that used for the section 122. However, if the data signals 104 from catheter 130 do not fall outside of the predetermined normal range for the blood pressure and/or flow parameters, such as when a cardiac event sensed by data signals 104 from sensors 109 is accompanied by a minimal hemodynamic response as determined by data signals 104 from the catheter 130, the CPU 112 will still display section 124 of waveform 120 in a manner similar or corresponding to section 122 of waveform 118. In this manner, the device 100 can still visually indicate or alert the observing individual to the abnormal data section 122 in waveform 118 with an indication of the corresponding normal data section 124 in waveform 120, as well as any other associated waveforms on the display 103, for monitoring and analysis purposes.

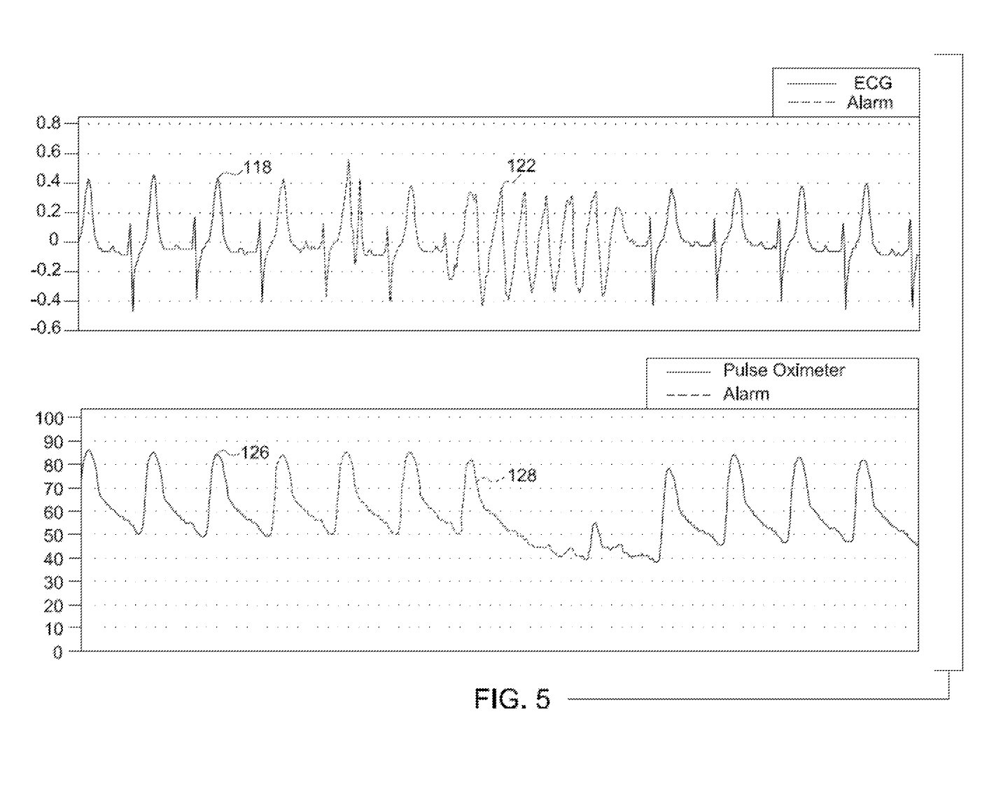

In still another exemplary embodiment shown in FIG. 5, the ABP waveform 120 can be substituted by a pulse oximeter waveform 126, which is displays blood pressure/flow data obtained from the pulse oximeter sensor 121. The data signals 104 received from the pulse oximeter sensor 121 are utilized by the CPU 112 to generate the waveform 126, as well as by the inbuilt algorithm to identify the section(s) 128 corresponding to any cardiac event measured by the ECG sensors 109 and illustrated in section 122 on the ECG waveform 118, whether or not these sections 128 are also outside of predetermined normal parameter ranges for the pulse oximeter 121 data signal 104.

In alternative embodiments, the enhanced visual identification provided to the sections 122,124 of the waveforms 118,120 shown on the display 102 can be utilized in conjunction with any other alarm notifications, such as audio or other visual notifications to further increase the efficiency and effectiveness of diagnostic systems in manufacturing, medical and other industries which include the diagnostic and/or monitoring devices 100. Also, the device 100 can display any number or combination of incoming data signal representations or waveforms 118,120,126, as desired, including but not limited to two or more waveforms 118,120,126, such that the incoming data signals 104 represented by the waveforms 118,120,126 can be shown side-by-side for a direct comparison of the waveforms 118,120,126 and the sections 122,124,128 that may be present therein.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A monitoring device for providing information on data obtained from sensors operably connected to the device, the device comprising:
a) a central processing unit configured to receive incoming data signals from a first sensor concerning a first physiological parameter and from a second sensor concerning a second physiological parameter, and to compare the incoming data signals to predetermined ranges for the first and second physiological parameters to determine an alarm condition; and
b) a display operably connected to the central processing unit, the display including a display screen and configured to graphically represent the incoming data signals from both the first sensor and the second sensor relating to a determined alarm condition on the display screen in a visually distinct manner from a remainder of the incoming data signals from the first sensor and the second sensor.

2. The device of claim 1 wherein the visually distinct manner comprises graphically representing incoming data signals relating to the alarm condition in a first color and representing the remainder of the incoming data signals in a second color.

3. The device of claim 2 wherein the wherein the first color is selected from the group consisting of a background color for the incoming data signals relating to the alarm condition and a color of a graphical representation of the incoming data signals relating to the alarm condition.

4. The device of claim 1 wherein the predetermined range is a frequency range for the incoming data signals.

5. The device of claim 1 wherein the alarm condition is an arrhythmia.

6. The device of claim 1 wherein the central processing unit is configured to graphically represent the incoming data signals from both the first sensor and the second sensor relating to a determined alarm condition on the display screen in a visually distinct manner from a remainder of the incoming data signals from the first sensor and the second sensor when the alarm condition is determined from the incoming data signals from only the first sensor.

7. The device of claim 6 wherein the central processing unit is configured to graphically represent the incoming data signals from the second sensor relating to a determined alarm condition on the display screen in a visually distinct manner from a remainder of the incoming data signals from the second sensor based on a correlation between the determined alarm condition and the incoming data from the second sensor.

8. The device of claim 1 wherein the correlation is a temporal correlation between the determined alarm condition and the incoming data signal from the second sensor.

9. The device of claim 1 wherein the first sensor comprises an ECG sensor.

10. The device of claim 1 wherein the second sensor is a sensor for determining blood pressure or blood flow.

11. The device of claim 10 wherein the second sensor is selected from the group consisting of an invasive pressure catheter and a pulse oximieter.

12. A method for identifying incoming data signals illustrated on a display screen falling outside of a predetermined range for the incoming data signals, the method comprising the steps of:
a) providing a monitoring device including a central processing unit configured to receive incoming data signals from a first sensor concerning a first operating parameter and from a second sensor concerning a second operating parameter, and to compare the incoming data signals to predetermined ranges for the first and second parameters to determine an alarm condition, and a display operably connected to the central processing unit and including the display screen;
b) comparing the incoming data signals from the first and second sensors with the predetermined ranges for the incoming data signals to determine the alarm condition;
c) identifying the incoming data signals from the first and second sensors relating to a determined alarm condition on the display screen in a visually distinct manner from a remainder of the incoming data signals from the first and second sensors.

13. The method of claim 12 wherein the step of identifying the incoming data signals on the display screen comprises:
a) representing the incoming data signals relating to the determined alarm condition in a first color; and
b) representing the remainder of the incoming data signals in a second color.

14. The method of claim 13 wherein the step of representing the incoming data signals relating to the determined alarm condition comprises displaying an aspect of the incoming data signals relating to the determined alarm condition in the first color, the aspect selected from the group consisting of a background and a graph for the incoming data signals relating to the determined alarm condition.

15. The method of claim 14 wherein the alarm condition is determined only from incoming data signals from the first sensor.

16. The method of claim 14 wherein the step of identifying the incoming data signals relating to the determined alarm condition comprises:
a) identifying sections of the incoming data signals from the first sensor causing the determined alarm condition on the display screen in a visually distinct manner; and
b) identifying sections of the incoming data signals from the second sensor having a correlation the incoming data signals from the first sensor causing the determined alarm condition in a visually distinct manner.

17. The method of claim 16 wherein the correlation is a temporal correlation.

18. A medical monitoring device for providing information about patient operably connected to the device, the device comprising:
a) an electronic storage medium in which a predetermined range of values for parameters of the patient to be monitored is stored;
b) a central processing unit operably connected to the electronic storage medium and configured to receive incoming data signals concerning the parameters of the patient and to compare the incoming data signals to the predetermined ranges for the parameters to determine an alarm condition;
c) one or more sensors operably connected to the central processing unit and adapted to be connected to the patient to obtain and transmit the incoming data signals on the parameters to be monitored from the patient to the central processing unit; and
d) a display operably connected to the central processing unit, the display including a display screen and configured to graphically represent the incoming data signals in waveforms presented on the display screen wherein sections of the waveforms containing incoming data signals relating to a determined alarm condition are presented on the display screen in a visually distinct manner from the remainder of the waveforms.

19. The medical monitoring device of claim 18 wherein sections of all waveforms corresponding to a section of one waveform containing incoming data signals relating to a determined alarm condition are presented on the display screen in a visually distinct manner from the remainder of the waveforms.

20. The medical monitoring device of claim 19 wherein sections of all waveforms corresponding in time to a section of one waveform containing incoming data signals relating to a determined alarm condition are presented on the display screen in a visually distinct manner from the remainder of the waveforms.
